(19) United States
(12) Patent Application Publication
(10) Pub. No.: US 2012/0039857 A1
(43) Pub. Date: Feb. 16, 2012

(54) SYSTEMS AND METHODS FOR CARDIAC TISSUE REPAIR

(75) Inventors: Rachel Smith, Sherman Oaks, CA (US); Linda Marbán, Beverly Hills, CA (US)

(73) Assignee: CAPRICOR, INC., Los Angeles, CA (US)

(21) Appl. No.: 13/263,315

(22) PCT Filed: Apr. 6, 2010
(86) PCT No.: PCT/US2010/030134

§ 371 Date: Oct. 6, 2011

Related U.S. Application Data
(60) Provisional application No. 61/167,025, filed on Apr. 6, 2009.

(51) Int. Cl. A61K 35/34; A61P 9/00

(52) U.S. Cl. 424/93.7

(57) ABSTRACT

The present application relates to cardiac stem cells and a method of using cardiac stem cells to repair damaged heart tissue. In one embodiment, cardiac stem cells, such as cardiosphere-derived cells and/or cardiospheres, can be seeded, embedded and/or cultured in a biomaterial or matrix made from, for example, a hydrogel, that is subsequently administered to a subject to repair damaged heart tissue.

### CROSS-REFERENCE TO RELATED APPLICATION

This application claims benefit of U.S. Provisional Application No. 61/167,025, filed Apr. 6, 2009, the disclosure of which is expressly incorporated by reference herein.

### BACKGROUND

1. Field of the Invention

The present application relates generally to systems and methods for repair of damaged cardiac tissue and/or regeneration of healthy cardiac tissue. In particular, isolated cardiac cells are cultured and may be seeded, embedded or otherwise incorporated into a biomaterial or synthetic graft that is administered to damaged cardiac tissue.

2. Description of the Related Art

Coronary heart disease is presently the leading cause of death in the United States, taking more than 650,000 lives annually. According to the American Heart Association, 1.2 million people suffer from a heart attack (or myocardial infarction, MI) every year in America. Of those who survive a first MI, many (25% of men and 38% of women survivors) will still die within one year of the MI. Currently, 16 million Americans are MI survivors or suffer from angina (chest pain due to coronary heart disease). Coronary heart disease can deteriorate into heart failure for many patients. 5 million Americans are currently suffering from heart failure, with 550,000 new diagnoses each year. Regardless of the etiology of their conditions, many of those suffering from coronary heart disease or heart failure have suffered permanent heart tissue damage, which often leads to a reduced quality of life. Accordingly, it is highly desirable to provide a method of treating or repairing damaged or diseased heart tissue.

### SUMMARY

Cell therapy, the introduction of new cells into a tissue in order to treat a disease, represents a possible method for repairing or replacing diseased tissue with healthy tissue. Stem cells are pluripotent cells capable of differentiating into a variety of different cell types. Embryonic stem cells, which are typically derived from an early stage embryo, have the potential to develop into any type of cell in the body. In some instances, unplanned growth of one cell type in a distinct type of tissue may result in the formation of teratomas. In contrast, adult stem cells generally develop into cell types related to the tissue from which the stem cells were isolated.

Use of embryonic stem cells in a clinical setting is often problematic because embryonic stem cells are typically allogeneic to a patient, as the embryonic stem cells rarely originate from that patient. As a result, rejection of transplanted embryonic stem cells may be a significant concern. Likewise, the pluripotency of embryonic stem cells does not guarantee differentiation into cells related to the target tissue. In contrast, adult stem cells taken from the patient and subsequently reintroduced into the same patient will generally not be rejected. Further, because adult stem cells generally develop into related cell types, the risk that the adult stem cells will develop into undesired cell types can be reduced by taking adult stem cells from the tissue that is to be treated or repaired. However, there still remains a need in the art for effective and efficient administration of adult stem cells into the heart to treat the cardiac tissue damage that results from an adverse cardiac event.

In one embodiment of the invention, a method of repairing or regenerating cardiac tissue is provided. The cardiac tissue is damaged or otherwise compromised in several embodiments because of an adverse cardiac event including, but not limited to, myocardial infarction, ischemic cardiac tissue damage, congestive heart failure, aneurysm, atherosclerosis-induced events, cerebrovascular accident (stroke), and coronary artery disease.

In several embodiments, the invention comprises a method of facilitating targeted delivery of cardiac stem cells and enhancing engraftment of the cells to repair or regenerate cardiac tissue. In one embodiment, the method comprises obtaining cardiosphere-derived cells, combining the cells with a biocompatible hydrogel comprising cross-linked hyaluronan to generate a matrix, and injecting the matrix into a subject to repair or regenerate cardiac tissue. In one embodiment, the matrix is adapted to initially retain the cells upon injection and to subsequently allow release and migration of the cells from the matrix to a targeted cardiac tissue. In one embodiment, the matrix is adapted to promote the survival of the cells. In one embodiment, the matrix facilitates the targeted delivery of the cells to the targeted cardiac tissue over time in vivo by reducing extraneous migration of the cells to non-targeted locations, and enhance engraftment of the cells into the targeted tissue, thereby repairing or regenerating the cardiac tissue. The cells incorporated into the matrix may be obtained from healthy mammalian non-embryonic cardiac tissue, though in some embodiments, embryonic tissue may be used. In one embodiment, the cells are combined with the matrix in a concentration of from about 1000 to about 10000 cells per microliter of matrix, from about 3000 to about 5000 cells per microliter of matrix, or from about 1000 to about 1250 cells per microliter of matrix.

In one embodiment, hydrogel further comprises collagen. In some embodiments, the collagen is thiolated collagen. In some embodiments the collagen results in preferential attachment of cardiosphere-derived cells to the matrix.

In one embodiment, the matrix facilitates the retention of injected cells at the site of injection in the targeted cardiac tissue for at least twenty-four hours post-injection.

In one embodiment, the matrix is adapted to promote the survival of about 60% of the cells within the matrix for a period of at least 72 hours.

In one embodiment, the matrix is adapted to allow maximal migration rates of the cells out of the matrix to the targeted cardiac tissue for a period of at least about twenty-four hours post-injection.

In one embodiment, the sample of healthy mammalian cardiac tissue is obtained from the subject (the transfer of cells to the subject is autologous). In one embodiment, the sample of healthy mammalian cardiac tissue is obtained from a mammal other than the subject (the transfer of cells to the subject is allogeneic). In one embodiment, the sample of healthy mammalian cardiac tissue is obtained from a mammal identical to the subject (the transfer of cells to the subject is syngeneic). In one embodiment, the sample of healthy mammalian cardiac tissue is obtained from a mammal of a different species than the subject (the transfer of cells to the subject is xenogeneic).

In one embodiment, injection of the cells comprises injection of the matrix to the heart of the subject. In one embodiment, the injection is performed using a catheter. The catheter may be dimensioned to limit backflow from the needle track post-injection. The catheter may be used in conjunction with an electromechanical mapping system.

In one embodiment, the biocompatible hydrogel comprising cross-linked hyaluronan is crosslinked with polyethylene glycol diacrylate. The polyethylene glycol diacrylate may combined with the hyaluronan in ratios ranging from between about 16 parts hyaluronan to 1 part polyethylene glycol diacrylate to about 1 part hyaluronan to 1 part polyethylene glycol diacrylate. In some embodiments, the polyethylene glycol diacrylate is combined with the hyaluronan in a ratio of 4 parts hyaluronan to 1 part polyethylene glycol diacrylate.

In some embodiments, the ratio of hyaluronan to polyethylene glycol diacrylate results in a gelation time of between approximately 1 minute to approximately 60 minutes. In one embodiment the ratio of hyaluronan to polyethylene glycol diacrylate results in a gelation time of approximately 20 minutes.

In one embodiment, the cells incorporated into the matrix express one or more markers selected from the group consisting of c-Kit, CD105, Sca-1, CD34, and CD31.

In several embodiments, the method provided is directed to regeneration of cardiac damaged by an adverse cardiac event. The adverse cardiac event may be a myocardial infarction, ischemic cardiac tissue damage, congestive heart failure, aneurysm, atherosclerosis-induced events, cerebrovascular accident (stroke), or coronary artery disease.

In several embodiments of the invention, a system for repair or regeneration of cardiac tissue is provided. In one embodiment, the system comprises isolated cardiosphere-derived cells, a cell-containing matrix comprising a biocompatible hydrogel comprising cross-linked hyaluronan, and a delivery device. The cardiosphere-derived cells may be isolated from healthy mammalian non-embryonic cardiac tissue, or alternatively, from embryonic tissue. The isolated cardiosphere-derived cells are incorporated into the cross-linked hyaluronan to generate the matrix, which is biocompatible and suitable for in vivo administration to the cardiac tissue of a subject.

In one embodiment of the system, the matrix is adapted to initially retain the cells upon administration, to promote the survival of the cells, and to subsequently allow release and migration of the cells from the matrix to a targeted cardiac tissue. The matrix facilitates the targeted delivery of the cells to the cardiac tissue over time in vivo by reducing extraneous migration of the cells to non-targeted locations and the matrix enhances engraftment of the cells into the cardiac tissue.

In one embodiment of the system, the cardiosphere-derived cells are incorporated into the biocompatible biomaterial in a concentration of about 1000 to 10000 cells per microliter of biomaterial.

In one embodiment, the system is provided for the transfer of autologous cells to the subject. In one embodiment, the system is provided for the transfer of allogeneic cells to the subject. In one embodiment, the system is provided for the transfer of syngeneic cells to the subject. In one embodiment, the system is provided for the transfer of xenogeneic cells to the subject.

In one embodiment, the hydrogel of the system further comprises collagen. In one embodiment the collagen is thiolated collagen.

In one embodiment of the invention, the use of a biocompatible stem cell-containing matrix for the repair or regeneration of damaged or diseased cardiac tissue is provided. The matrix may comprise a hydrogel comprising cross-linked hyaluronan combined with cardiosphere-derived cells. The matrix is suitable for administration to a subject having damaged or diseased cardiac tissue, and is adapted to initially retain the cells upon administration and to subsequently allow release and migration of the cells from the matrix to the targeted cardiac tissue. In one embodiment, the matrix promoted the survival of the cells. The matrix reduces extraneous migration of the cells to non-targeted locations and enhances engraftment of the cells into the heart, thereby repairing or regenerating the cardiac tissue.

In one embodiment, the use of the matrix includes cardiosphere-derived cells that are obtained from a sample of healthy mammalian non-embryonic cardiac tissue.

In one embodiment, the hydrogel used further comprises collagen. In one embodiment, the collagen is thiolated collagen. In some embodiments, use of matrix comprising collagen results in preferential attachment of cardiosphere-derived cells to the matrix.

In one embodiment, use of the matrix facilitates the retention of injected cells at the site of administration in the targeted cardiac tissue for at least twenty-four hours post-injection. In one embodiment, use of the matrix promotes the survival of about 60% of the cells within the matrix for a period of at least 72 hours. In one embodiment, use of the matrix allows maximal migration rates of the cells out of the matrix to the targeted cardiac tissue for a period of at least about twenty-four hours post-administration.

In one embodiment, the use of the matrix is for the transfer of autologous cells to the subject. In one embodiment, the use of the matrix is for the transfer of allogeneic cells to the subject. In one embodiment, the use of the matrix is for the transfer of syngeneic cells to the subject. In one embodiment, the use of the matrix is for the transfer of xenogeneic cells to the subject.

In one embodiment, the use of the matrix is includes cardiosphere-derived cells expressing one or more markers selected from the group consisting of c-Kit, CD105, Sca-1, CD34, and CD31.

In one embodiment, the method repairing of regenerating cardiac tissue comprises obtaining a sample of healthy mammalian cardiac tissue and fragmenting the cardiac tissue in vitro to obtain a plurality of cardiac tissue fragments. The cardiac tissue may be embryonic, however, in several embodiments, the cardiac tissue is non-embryonic. Adult cardiac tissue is used in many embodiments. After fragmentation, the cardiac tissue fragments are allowed to adhere to a solid support and are cultured in a culture medium having one or more nutrients. The tissue fragments are of a size sufficient to allow the diffusion of the nutrients present in the medium to the tissue fragments. The tissue fragments are cultured in the media until one or more phase-bright cells form. The phase-bright cells are harvested and cultured on a treated substrate to generate cardiac stem cells. Cardiac stem cells include cardiospheres, cardiosphere-derived cells, or both. The cardiac stem cells are combined, or otherwise coupled, with a biomaterial to generate a matrix. The matrix, in several embodiments, permits survival and migration of the cardiac stem cells over time in vivo. In several embodiments, the matrix is administered to a patient to repair or regenerate cardiac tissue.

In one embodiment of the invention, a system for repair or regeneration of cardiac tissue is provided. In one embodiment, the system comprises isolated mammalian cardiac tissue that is cultured to yield cardiac stem cells. The cardiac stem cells comprise cardiospheres and/or cardiosphere-derived cells. The system further comprises a biocompatible biomaterial, such as hyaluronan, alginate, and/or fibrin. The cardiac stem cells are incorporated into the biocompatible biomaterial in a concentration of about 500 to about 100,000 cells per microliter of biomaterial. In some embodiments, about 1000 to about 10,000 cells per microliter are used. The biomaterial and cardiac stems cells are combined to form a matrix that is configured to release the cardiac stems cells into a damaged heart to repair or regenerate cardiac tissue upon administration to a patient.

In several embodiments described herein, the biomaterial includes, but is not limited to, hyaluronan, alginate, fibrin, or combinations of at least two or three biomaterials thereof.

In several embodiments described herein, the cardiospheres are multicellular aggregates and comprise a mixed population of cells. The mixed population comprises stem cells. The cardiospheres are, in some embodiments, weakly adherent or non-adherent to the treated substrate.

In several embodiments described herein, the number of cardiac stem cells combined with biomaterial is from about 1000 to about 10,000 cells per microliter of biomaterial. In some embodiments, about 100 to about 1,000,000 cells per microliter are used (e.g., about 500, 1000, 5,000, 10,000, 50,000, 100,000, 250,000, 500,000).

In several embodiments described herein, the healthy mammalian cardiac tissue is obtained from and administered to (in the form of the matrix) the same subject. In other embodiments, the healthy tissue is obtained from one subject, and administered to a different subject. In some embodiments, the subject is a human. In other embodiments, the subject is a non-human mammal.

According to several embodiments, administration of the matrix to a subject involves the injection of the matrix. In other embodiments, catheter-based delivery systems are used. In one embodiment, the matrix is delivered to the heart. In other embodiments, the matrix is delivered proximate to the heart. Delivery routes include, but are not limited to, intracoronary, intravascular or intracardiac. Delivery may be accomplished with specific injection site guidance in several embodiments. In one embodiment, NOGA is employed.

In several embodiments, the biomaterial comprises one or more cross-linking agents, which may in some embodiments, increase the viscosity of the matrix. The cross-linker is used in a concentration maximizes the migration of the cells out of the matrix and into the damaged cardiac tissue in one embodiment. Polyethylene glycol diacrylate is used in several embodiments as the cross-linker. In several embodiments, collagen is used.

### BRIEF DESCRIPTION OF THE FIGURES

FIG. 1 depicts a schematic for isolation of cardiac stem cells from a sample (e.g., biopsy) of cardiac tissue according to several embodiments discussed herein.

FIG. 2a-f depicts the morphology of CDCs embedded in hyaluronan in culture at multiple time points.

FIG. 3a-b depicts the percentage of CDCs surviving after being embedded in hyaluronan at various concentrations and cultured for 7 days.

FIG. 3c-e depicts the fluorescent microscopy used to analyze live versus dead cells.

FIG. 4a-l depicts the morphology of CDCs embedded in serum or collagen supplemented hyaluronan in culture at multiple time points.

FIG. 5a depicts the percentage of CDCs surviving after being embedded in serum or collagen supplemented hyaluronan and cultured for 7 days.

FIG. 5b-g depicts the fluorescent microscopy analysis of live versus dead cells.

FIG. 6 depicts the survival of various concentrations of CDCs cultured in hyaluronan alone or collagen-supplemented hyaluronan after 72 hours.

FIG. 7a depicts the survival of CDCs cultured in hyaluronan alone or collagen-supplemented hyaluronan after 1 week.

FIG. 7b-g depicts the fluorescent microscopy analysis of live versus dead cells.

FIG. 8a-b depicts the migration and migration rate of CDCs out of various hydrogels over 48 hours.

FIG. 8c-e depicts bright field visualization of in vitro migration of CDCs.

FIG. 9a depicts the percentage of in vivo engraftment of CDCs released from hydrogels.

FIG. 9b-c depicts fluorescent visualization of engrafted of CDCs.

### DETAILED DESCRIPTION

In several embodiments described herein, methods of harvesting, culturing, preparing, and introducing cardiac cells into a patient who has previously suffered an adverse cardiac event result in repair of damaged cardiac tissue in the patient. In other embodiments, the introduced cells effect a regeneration of healthy cardiac tissue, which functionally replaces the tissue affected by the adverse cardiac event.

Frequently, first generation cell therapy studies and clinical trials are designed to evaluate the safety of the intervention and therefore employ non-invasive delivery routes (e.g., intracoronary or intravenous infusion). In conjunction with use of saline as a carrier, such studies often yield low cell retention rates and decreased incidence of long-term persistence of the transplanted cells. This may be due to a variety of factors, including cell washout and/or low cell survival rates in the delivery media. Thus, in an effort to more fully develop the potential of cell therapies, several embodiments described herein are directed to more efficient means of delivering cells in order to maximize short and long-term engraftment.

In several embodiments, cells (such as cardiospheres and cardiosphere-derived cells) are coupled to a biomaterial to form a matrix. The matrix, in several embodiments, is particularly advantageous because, by keeping the cells together, permits the delivery of a bolus of concentrated cells that remain together for a desired time once administered. In some embodiments, the matrix facilitates cell stability and survival rate. In several embodiments, the matrix improves the retention of the cells in the cardiac tissue. In some embodiments, the matrix enhances cell migration to the damaged regions of cardiac tissue. In several embodiments, cell survival is increased as a result of the cellular interaction with the matrix. In several embodiments, cell survival is increased as a result of paracrine interactions with other cells, reduced toxin production or accumulation, or increased structural support, or combinations thereof. In further embodiments, the matrix permits a smaller, yet equally or more efficacious, therapeutic dose of cells to be delivered to the damaged tissue. In still other embodiments, the matrix enhances cell engraftment and differentiation, which may result in improved cardiac function. In some embodiments, cells that have differentiated in the matrix express one or more cardiac differentiation markers, such as cardiac troponin I, Nkx 2.5, alpha-sarcomeric actin. Other cardiac differentiation markers are detected in some embodiments. In some embodiments, cells that have differentiated in the matrix express one or more endothelial differentiation markers, such as CD31 or von Willebrand factor. Other endothelial differentiation markers are detected in some embodiments. In some embodiments, cells that have differentiated in the matrix express one or more smooth muscle markers, such as alpha-smooth muscle actin. Other smooth muscle markers are detected in some embodiments. In several such embodiments, matrix-cardiac stem cell interactions occur that reduce or block apoptosis pathways. In other embodiments, the matrix reduces the cytotoxic impact of the damaged target tissue on the cells combined with the matrix.

As used herein, the term “adverse cardiac event” shall be given its ordinary meaning and shall also be read to include, but not be limited to myocardial infarction, ischemic cardiac tissue damage, congestive heart failure, aneurysm, atherosclerosis-induced events, cerebrovascular accident (stroke), and coronary artery disease.

As used herein, the term “matrix” shall be given its ordinary meaning and shall also be read to include, but not be limited to biological and synthetic materials that can support living cells. A matrix may comprise, for example, hyaluronan, alginate, fibrin or combinations thereof. A matrix may comprise biograft material or synthetic graft material. A matrix can be liquid, gelatinous or solid. A matrix may be embedded or seeded with, for example, cardiospheres, cardiosphere-derived cells, cardiosphere-forming cells, phase bright cells, stem cells, or other cells, or combinations thereof. A matrix may comprise a scaffold or platform. The terms matrix and biomaterial are used interchangeably herein.

In several embodiments, cells are incorporated into biomaterial sheets or within sponge or foam-like structures that may be applied as a patch to the surface of the heart. In several embodiments, the matrix (or biomaterial) used comprises a gel or hydrogel, allowing cells embedded within to be injected or infused into the heart or applied as a paint or glue to the surface of the heart. In still other embodiments, a cross-linker may be used to alter the viscosity of a hydrogel such that the matrix has characteristics that range from substantially fluid in nature to that of a flexible solid.

### Harvesting Donor Cardiac Tissue

Donor cardiac tissue can be harvested from a patient by obtaining small amounts of heart tissue through, for example, a biopsy. While a typical human adult heart weighs about 200 to 300 g, sufficient amounts of cardiac cells can be obtained from cardiac tissue samples of about 1 mg to about 50 mg. In some embodiments, the mass of the cardiac tissue sample is about 25 mg or less. The tissue sample may be obtained from a variety of locations in the heart, including but not limited to, the crista terminalis, the right ventricular endocardium, the right ventricular septum, the septal or ventricle wall, the atrioventricular groove and the right and left atrial appendages. Some of these locations have been identified as being relatively rich in cardiac stem cells. In some embodiments, donor cardiac tissue is obtained during a surgical procedure, such as bypass surgery. In other embodiments, donor tissue is obtained during a percutaneous endomyocardial biopsy procedure. Tissue may be obtained from embryonic or non-embryonic sources. Non-embryonic sources are preferred for several embodiments. In some embodiments, stem cells taken from a patient's own heart are administered back to the same patient (an autologous transfer). In other embodiments, stem cells taken from a donor are administered to a (non-donor) recipient (an allogeneic transfer).

A biopsy may be obtained, for example, by using a percutaneous bioptome as described in further detail in International Publications WO 2006/052925 to Marbán et al. and WO 2006/052927 to Marbán, both of which are hereby incorporated by reference in their entireties. Although a conventional bioptome may be used to obtain the tissue sample, conventional bioptomes are generally only able to collect samples from a limited number of locations in the heart due to their stiffness. Accordingly, use of a bioptome that includes a relatively flexible catheter and a means for steering will allow the surgeon to collect heart tissue from a wider variety of locations.

According to several embodiments, percutaneous endomyocardial biopsy specimens are harvested using the following procedure. Under local anesthesia, a guide catheter is introduced into a vein, such as the jugular vein, in the patient's neck if tissue samples are to be taken from the right ventricle. Alternatively, the guide catheter can be introduced into an artery if tissue samples are to be taken from the left ventricle. The guide catheter is guided to the heart with the aid of visualization provided by a standard imaging technique, such as fluoroscopy. Once the guide catheter is in place, the bioptome can be introduced into the guide catheter and threaded to the heart. Once the bioptome is within the heart, the flexible distal end of the bioptome can be manipulated by the surgeon to extract a tissue sample from the desired location. The bioptome can be removed from the patient so that the tissue sample can be retrieved and then the bioptome can be reintroduced so that another sample can be taken from the same or different location. In another embodiment, the bioptome can extract multiple samples before being withdrawn, thereby reducing the time needed to collect the tissue samples.

### Culturing Cardiospheres and Cardiosphere-Derived Cells

Adult human hearts have reservoirs of cardiac stem cells. In some embodiments, methods described herein yield a mixed population of cells that comprises, for example, stem cells, cardiac cells, and/or vascular cells. The mixed population of cells expresses various stem cell markers. In some embodiments, the vascular cells express at least one of flk-1 and CD31. In other embodiments, cardiac stem cells may be identified in adult mammals by several stem cell-related markers including CD34, Sca-1, c-Kit, and CD105. In several embodiments, the stem cells do not express one or more of the stem cell markers identified above. In vitro, cardiac stem cells are clonogenic and can give rise to immature cardiomyocytes (heart muscle cells) and endothelial and smooth muscle cells (blood vessel components). Cardiac stem cells are also identifiable by their ability to form cardiospheres, and cardiosphere-derived cells (CDCs) in culture, as described in WO 2006/052925 to Marbán et al. and US 2007/0020758 to Giacomello et al., which are hereby incorporated by reference in their entireties. In some embodiments, cardiospheres, and subsequently CDCs, are isolated for use in repairing damaged heart tissue, as these cells are resident in the heart and are genetically pre-programmed to reconstitute all cardiac lineages. Several embodiments of the invention are particularly advantageous because, when implanted, the cells (e.g., cardiospheres, CDCs) give rise to all necessary components of cardiac tissue, without producing undesired tissue growth (e.g., teratomas).

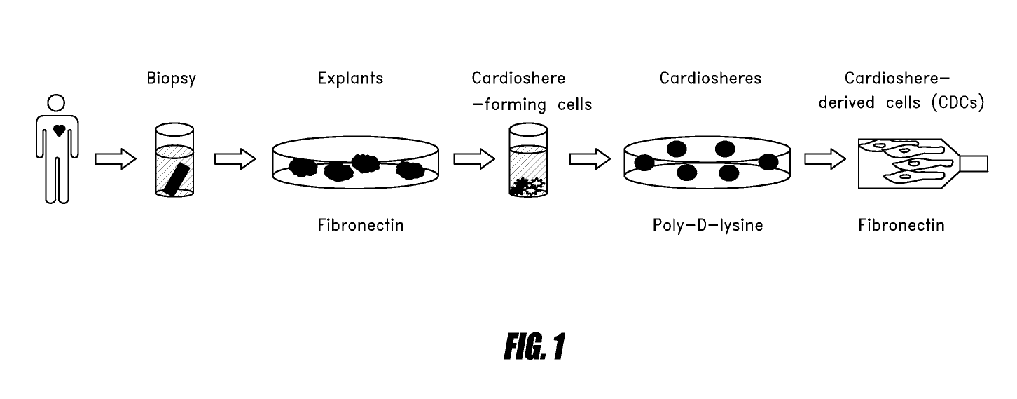

In one embodiment of the invention, cardiospheres and CDCs are isolated as according to the schematic in FIG. 1. Briefly, cardiac tissue samples are weighed, cut into small fragments and cleaned of gross connective tissue, and washed in a sterile solution, such as phosphate-buffered saline. In some embodiments, the tissue fragments are at least partially digested with protease enzymes such as collagenase, trypsin, and the like. In several embodiments, the digested pieces are placed in primary culture as explants on sterile tissue culture dishes with a suitable culture media. The digested pieces of tissue range in size from about 0.1 mm to about 2.5 mm. In several embodiments, the digested pieces of tissue range 0.25 mm to about 1.5 mm. Smaller or larger pieces of tissue can be used in other embodiments. In several embodiments, the digested pieces of tissue range 0.1-0.25 mm, 0.25-0.5 mm, 0.5-1 mm, 1-1.25 mm, 1.25-1.75 mm, 1.75-2.25 mm, 2.25-2.5 mm, and overlapping ranges thereof. In some embodiments, methods according to several embodiments of the invention are particularly advantageous because they are compatible with the use of a smaller sample of initial cardiac tissue, such as sample obtained through a minimally-invasive biopsy procedure. For example, the initial tissue sizes ranges from about 1.0 to 3.0 mm in diameter in several embodiments, including about 1.5, 1.7, 1.9, 2.1, 2.3, 2.5, 2.7, 2.8 or 2.9 mm in diameter. In one embodiment, the tissue culture dish and culture media are selected so that the tissue fragments adhere to the tissue culture plates. In some embodiments, the tissue culture plates are coated with fibronectin or other extracellular matrix (ECM) proteins, such as collagen, elastin, gelatin and laminin, for example. In other embodiments, the tissue culture plates are treated with plasma. In several embodiments, the dishes are coated with fibronectin at a final concentration of from about 10 to about 50 μg/mL. In still other embodiments, the fibronectin dishes are coated with fibronectin at a final concentration of from about 20 to 40 μg/mL, with still other embodiments employing a final fibronectin concentration of about 25 μg/mL.

In several embodiments, the base component of the complete explant medium comprises Iscove's Modified Dulbecco's Medium (IMDM). In some embodiments, the culture media is supplemented with fetal calf serum (FCS) or fetal bovine serum (FBS). In several embodiments, the media is supplemented with serum ranging from 5 to 30% v/v. In other embodiments, the culture media is serum-free and is instead supplemented with specific growth factors or hydrolyzed plant extracts. In other embodiments, the media is further supplemented with antibiotics, essential amino acids, reducing agents, or combinations thereof. In one embodiment, the complete explant medium comprises IMDM supplemented with about 20% fetal bovine serum, about 50 μg/mL gentamicin, about 2 mM L-glutamine, and about 0.1 mM 2-mercaptoethanol. In some embodiments, the explant media is changed every 2-4 days while the explants culture.

The tissue explants are cultured until a layer of stromal-like cells arise from adherent explants. This phase of culturing is further identifiable by small, round, phase-bright cells that migrate over the stromal-cells. In several embodiments, the explants are cultured until the stromal-like cells grow to confluence. At or before that stage, the phase-bright cells are harvested. In several embodiments, phase-bright cells are harvested by manual methods, while in others, enzymatic digestion, for example trypsin, is used. The phase-bright cells may be termed cardiosphere-forming cells, and the two phrases are used interchangeably herein.

Cardiosphere-forming cells may then be seeded on sterile dishes and cultured in cardiosphere media. In several embodiments, the dishes are coated with poly-D-lysine, or another suitable natural or synthetic molecule to deter cell attachment to the dish surface. In other embodiments, for example, laminin, fibronectin, poly-L-orinthine, or combinations thereof may be used.

In several embodiments, the base component of the cardiosphere medium comprises Iscove's Modified Dulbecco's Medium (IMDM). In some embodiments, the culture media is supplemented with fetal calf serum (FCS) or fetal bovine serum (FBS). In several embodiments, the media is supplemented with serum ranging from 5 to 30% v/v. In other embodiments, the culture media is serum-free and is instead supplemented with specific growth factors or hydrolyzed plant extracts. In several other embodiments, the media is further supplemented with antibiotics, essential amino acids, reducing agents, or combinations thereof. In one embodiment the cardiosphere medium comprises IMDM supplemented with about 10% fetal bovine serum, about 50 ng/mL gentamicin, about 2 mM L-glutamine, and about 0.1 mM 2-mercaptoethanol.

According to one embodiment, cardiospheres will form spontaneously during the culturing of the cardiosphere forming cells. Cardiospheres are recognizable as spherical multicellular clusters in the culture medium. Cells that remain adherent to the poly-D-lysine-coated dishes are discarded. In several embodiments, the cardiospheres are collected and used to seed a biomaterial or synthetic graft. In other embodiments, the cardiospheres are further cultured on coated cell culture flasks in cardiosphere-derived stem cell (CDC) medium.

In some embodiments used to culture cardiospheres into CDCs, the culturing flasks are fibronectin coated, though in other embodiments other cellular attachment promoting coatings are employed. The cultured cardiospheres attach to the surface of the flask and are expanded as a monolayer of CDCs. CDC medium comprises IMDM, and in several embodiments is supplemented with fetal calf serum (FCS) or fetal bovine serum (FBS). In some embodiments, the media is supplemented with serum ranging from 5 to 30% v/v. In other embodiments, the culture media is serum-free and is instead supplemented with specific growth factors or hydrolyzed plant extracts. In several other embodiments, the media is further supplemented with antibiotics, essential amino acids, reducing agents, or combinations thereof. In one embodiment, the CDC medium comprises IMDM supplemented with about 10% fetal bovine serum, about 2 mM L-glutamine, and about 0.1 mM 2-mercaptoethanol. CDCs may be repeatedly passaged by standard cell culture techniques and in several embodiments are harvested and used to seed a biomaterial or synthetic graft.

Although CDCs are used in some embodiments to seed a biomaterial or synthetic graft and some embodiments employ cardiospheres, in still other embodiments cardiac stem cells can be directly used to seed a biomaterial or synthetic graft. In some embodiments, the cells used are allogeneic to the recipient, while in others, the cells are autologous. In yet other embodiments, all the cells obtained from the biopsied tissue sample are used to seed the biomaterial or synthetic graft. In other embodiments, a subpopulation of the cells obtained from the biopsied tissue sample, including the cardiac stem cells and at least one other cell type, are used to seed the biomaterial or synthetic graft. The at least one other cell type can be any combination of cell types from the following non-inclusive list: endothelial cells, smooth muscle cells, fibroblasts, macrophages and other noncardiomyocytes.

### Biomaterials as Cell Carriers

The efficacy of cardiospheres and CDC transplantation can be improved, according to several embodiments, by embedding, seeding, or otherwise incorporating cardiospheres and/or CDCs within or onto various biocompatible biomaterials. As used herein, the term “biocompatible” shall be given its ordinary meaning and shall also include the ability of biomaterial to perform its desired function with respect to repair or regeneration of cardiac tissue, without eliciting any undesirable local or systemic effects in the recipient or beneficiary of the repair or regeneration of cardiac tissue.

Synthetic biocompatible polymers include, but are not limited to, biodegradable polymers such as polylactic acid (PLA), polyglycolic acid (PGA), poly(lactic-co-glycolic acid) (PLGA), polycaprolactone (PCL) and a variety of polycarbonate derivatives, and combinations thereof. Non-degradable biocompatible polymers include, for example, poly(ethylene oxide) (PEO), poly(ethylene glycol) (PEG) and poly(ethylene-co-vinyl acetate) (EVA).

Suitable biomaterials used in several embodiments include, but are not limited to, materials derived from biological sources. For example, ECM components including, among others, glycosaminoglycans, such as hyaluronan, proteoglycans and proteins may be used. ECM proteins include, for example, collagen, elastin, fibronectin, fibrin, gelatin and laminin. Other naturally occurring biopolymers and their derivatives, such as chitin, chitosan and alginate, may also be suitable.

### Hyaluronan

In several embodiments, the matrix comprises hyaluronan, alone or in combination with other materials. Hyaluronan is a glycosaminoglycan component of the ECM of all connective tissues, used in some embodiments as a biocompatible scaffold to deliver cardiospheres or CDCs to damaged cardiac tissue. In some embodiments, disulfide crosslinked hyaluronan hydrogels are created using thiolated hyaluronan derivatives and thiol-reactive crosslinkers such as polyethylene glycol diacrylate (PEGDA). In some embodiments, cells are incorporated during the crosslinking process where they attach and survive within the hydrogels. In some embodiments cells are incorporated into one component of the hydrogel prior to crosslinking. In some embodiments, the cells incorporated after the crosslinking process. The cells can be recovered, if needed, by enzymatic digestion of the hydrogels.

Gelation of hydrogels is time- and pH-dependent and may be further adjusted to the desired characteristics in any given embodiment by diluting the gel components. In several embodiments, dilution is used to reduce the reactive elements (thiolated hyaluronan derivatives and thiol-reactive crosslinkers) and yield a more liquid, and therefore injectable, hyaluronan cell-containing matrix. In those embodiments where the entirety of the biomaterial/graft is to dissipate over time after administration, hyaluronan-based hydrogels may be used, as they are biodegradable. The hydrogel biodegrades in vivo over the course of four to eight weeks due to the action of hyaluronidases produced naturally by cells. In several embodiments, hyaluronidases initiate the biodegradation process in vivo after about approximately one week. Hyaluronan is removed from the body by internalization and destruction in the cell lysosome or by draining into the vasculature followed by removal by the lymph nodes, liver, and kidneys.

In several embodiments, the intrinsic aversion of hyaluronan to cell attachment (due hyaluronon's hydrophilicity and cellular preferences for hydrophobic environments) may be overcome by blending other ECM proteins such as collagen with hyaluronan. In one such embodiment, thiolated collagen is covalently crosslinked to thiolated hyaluronan to create a cell compatible hydrogel. In some embodiments, the addition of collagen results in preferential cell attachment to the hydrogel. In some embodiments, the addition of collagen results in increased cell survival, migration, engraftment into target tissues, or combinations thereof.

In some embodiments, thiolated heparin is added to hydrogel in addition to, or in place of, collagen. Without wishing to be bound by theory, it is believed that the immobilized heparin mimics the heparan sulfate proteoglycans normally present in the ECM and binds ionically to growth factors, and allowing for their release over time. In several embodiments, the heparin binding of growth factors, either from the cells embedded in the biomaterial or the local tissue, leads to improved survival of transplanted cells and/or of resident cardiomyocytes. Supplementation of biomaterials with collagen alone may have similar effects in some embodiments. Improved cell survival may be due to increases in the local concentration of growth factors which promote cell function and vitality.

In other embodiments, gelatin (a heterogeneous mixture of water-soluble collagens with high, average molecular weights) in a thiol-modified format may be combined with hyaluronan, and PEGDA to create a hydrogel whose gelation time is approximately 20 minutes (when combined in an optional ratio of 2:2:1). Other ratios are used in several embodiments. Increasing the gelatin to hyaluronan ratio will increase gelation time in some embodiments. The eventual viscosity and gelation time are tailored, in several embodiments, to maximize the cell retention properties of the resulting matrix. Other ECM components (e.g., fibronectin, laminin, etc.) can be readily mixed into the hydrogel by adding an ECM solution to the hyaluronan solution. The matrix used according to several embodiments disclosed herein comprises about 25%-75% hyaluronan, 25%-75% collagen, and/or 5%-25% crosslinker. In one embodiment, the matrix comprises, consists or consists essentially of (i) hyaluronan and a crosslinker; (ii) collagen and a crosslinker; or (iii) hyaluronan, collagen and a crosslinker.

In several embodiments, the matrix provides a unique nutritional microenvironment. In some embodiments, the unique nutritional microenvironment is tailored to be optimal based on the target tissue that the matrix and cells will be delivered to. In some embodiments, the unique nutritional microenvironment is designed to maximize one or more of cell retention, cell proliferation, cell survival, cell migration, cell differentiation, or cell engraftment into the target tissue.

In several embodiments, the matrix provides a 3-dimensional scaffold which closely mimics the complex three dimensional cellular environments found in vivo. Thus, several embodiments are particularly advantageous because the unique nutritional and structure environment replicates or simulates the native environment of the incorporated cells, and in one embodiment, enhances cell viability.

In several embodiments, the components of are free from animal products (e.g., xeno-free) and fully defined. In several embodiments, the matrix further comprises additional components (e.g. nutrients, growth factors, or cross-linkers) that allow for a customized or tailored matrix, depending on the application. For example, some embodiments of the matrix incorporate on or more ECM proteins. Some embodiments of the matrix incorporate cell attachment factors. In some embodiments, certain additives protect growth factors from proteolysis in vivo and reduce the release rate of growth factors, thus creating a longer temporal presence of growth factors post-administration. As described herein, the addition of particular cross-linkers and their ratios allow control of the viscosity and elasticity of the resultant hydrogel. Moreover, in several embodiments, cells can be incorporated into the hydrogel in different manners depending on the application and the desired 3D environment (e.g. cell incorporation by encapsulation or top plating). Thus in several embodiments, the amount and type of growth factors incorporated, the amount and type of ECM proteins incorporated, and the resultant hydrogel stiffness or rigidity are all controllable. In one embodiment, the matrix incorporates agents that facilitate the migration of cells out of said matrix (e.g., agents based on chemoattractants/chemorepellents, hydrophobic/hydrophilic interactions, polarity, enzymes such as proteases, degradation molecules, signaling molecules, etc.).

In several embodiments, the cells incorporated into the matrix are cardiac stem cells. In some embodiments other stem cell are used (e.g., embryonic stem cells, umbilical cord blood stem cells, bone marrow derived stem cells, hepatic stem cells, and hepatic progenitor cells). In some embodiments, the hydrogel is particularly advantageous for hosting stem cells whose natural environment is rich in hyaluronic acid.

In several embodiments, the viscosity of the resultant hydrogel, in balance with in vivo chemoattractive forces control the retention of the cells. For example, a highly viscous matrix administered to a target tissue with little chemoattractivity will result in a high degree of cell retention and little migration of the cells from the matrix. In contrast, a less viscous matrix administered to a tissue that is rich with chemoattractants will result in less cell retention over time. In several embodiments, the viscosity of the matrix is tailored to ensure an initial higher degree of cell retention, such that cells are maintained in the targeted tissue administration site (e.g., not washed or pushed away). In such embodiments, the viscosity f the matrix does not thereafter inhibit migration of the cells into the desired target site.

As discussed above the controlled nature of the matrix allows for the migration time of the cells to be tailored to a specific target tissue site. In some embodiments, at or after 6 hours, over 10%, 25%, 50% or 75% of cells are released. In one embodiment, at or after 12 hours, over 25%, 50% or 75% of cells are released. In one embodiment, at or after 24 hours, over 25%, 50% or 75% of cells are released. In one embodiment, at or after 48 hours, over 50% or 75% of cells are released. In several embodiments, the released cells engraft in the targeted area. Although the targeted area may be cardiac tissue in some embodiments, repair or regeneration of other bodily organs is provided in some embodiments (e.g., skin or liver grafts).

### Alginate

In several embodiments, the matrix comprises alginate, alone or in combination with other materials. Alginate is a linear polysaccharide derived from brown algae. Alginate consists of β-D-mannuronate (M) and α-L-guluronate (G) monomers arranged homopolymerically, consecutively, randomly, or in an alternating fashion. Viscosity of the alginate may be varied for any particular embodiment by controlling the molecular weight of the M and G monomers, the alginate concentration, the polymerization temperature, and the presence and concentration of salts or ions. In several embodiments, M-rich alginate gels are used, which are softer and more fragile than G-rich alginate gels. In several such embodiments, the resulting M-rich alginate will also have a lower porosity compared to a G-rich alginate gel. In alternate embodiments, G-rich alginate is used for the biomaterial, thus making a more durable and “solid-like” biomaterial. In several embodiments, a low viscosity, and therefore injectable, alginate is prepared by polymerization in the presence of Ca⁺⁻ ions. Beneficial to several embodiments, alginate hydrogels are dissolved over time and the water-soluble alginate is excreted by the kidneys. In several embodiments, alginate is used to encapsulate cardiospheres or CDCs prior to administration to a patient.

### Fibrin

In several embodiments, the matrix comprises fibrin, alone or in combination with other materials. Fibrin is used in other arenas as a medical sealant. Fibrin is formulated from human plasma, and in several embodiments, prepared in an autologous manner. Fibrinogen, the fibrin precursor, along with a fibrinolysis inhibitor, is mixed with thrombin and calcium. The mixture remains a liquid for several seconds before solidifying into a gel fibrin matrix. In some embodiments, fibrin alone is used to deliver cardiospheres and/or CDCs to damaged cardiac tissue. In other embodiments, a preparation of fibrinogen, thrombin, calcium, and cardiospheres and/or CDCs are combined just prior to delivery. Alternative embodiments employ fibrinogen mixed with cardiospheres and/or CDCs and rely on target tissue thrombin to produce a fibrin-cell matrix upon delivery to the target cells of the heart.

Incorporation and Release of Cells from Biomaterials

In several embodiments, cell therapy is enhanced by the delivery of sufficient cell numbers to a target region of damaged cardiac tissue. Accordingly, in several embodiments, delivered cells survive in vivo until they diffuse out of the biomaterial into the cardiac tissue. As such, several embodiments vary the components of the delivered biomaterial to affect an optimal delivery of cells to the target tissue.

In several embodiments, the cardiospheres and/or CDCs are mixed with the biomaterial alone. In other embodiments, an appropriate cross-linking agent is added to the aforementioned cell-biomaterial mixture. The pre-mixing of the cells with the biomaterial allows the encapsulation of the cells within the biomaterial after cross linking in some embodiments.

In several embodiments, it is desirable to control the porosity of the biomaterial (e.g., hydrogel) and thus, the ability of nutrients and wastes to diffuse into and out of the hydrogel. As discussed above, several embodiments vary the relative amount of the appropriate cross-linking agent added to the biomaterial resulting in a decrease in average pore size and reduction in diffusion through the hydrogel. Conversely, alternative embodiments incorporate relatively smaller amounts of cross-linking agent, yielding increased pore size and diffusion through the hydrogel. Several embodiments achieve a balanced degree of structural integrity of the biomaterial and sufficient diffusion of nutrients and wastes.

Several embodiments include nutrients, additives and/or growth factors that are added to the biomaterial. Such additives may promote cell proliferation, cell differentiation or cell viability. Moreover, in addition to the composition of the biomaterial, additives may enhance cell retention. Still other embodiments do not necessitate additive to yield efficacious cell retention. Nutrients, additives and/or growth factors are not limited to those added in an in vitro setting, rather they may be released from the cells that are incorporated into the biomaterial or from the local target tissue into/onto which the cell-biomaterial composition is delivered. In addition, other nutrients such as glucose, insulin, pyruvate, amino acids, and growth factors are also incorporated into the biomaterial in some embodiments. Still other embodiments include serum supplementation of the biomaterial, with supplementation ranging from about 5-10% serum. In several embodiments, serum supplements the biomaterial at about 7.5%. In several embodiments, serum supplements the biomaterial in a range of about 5-7%, 6-8%, 7-9%, or 8-10%. In several other embodiments involving serum supplementation at 7.5%, the biomaterial is hyaluronan. In still other embodiments, the biomaterial is supplemented with one or more components associated with the ECM. In several of such embodiments, the biomaterial is supplemented with collagen. In some embodiments, collagen is added to the biomaterial in a range from about 0.2-0.6% of the final concentration, including 0.3%, 0.4%, and 0.5%. Lower or higher ranges may be used. In several embodiments, about 0.4% collagen is used to supplement hyaluronan to form a cell matrix.

In some embodiments, typically those made into more viscous biomaterial matrices, portions of the biomaterial can be selectively coated or be made to include growth factors and/or cytokines that promote, for example, cell migration, cell activation and/or cell differentiation. Coating or incorporation of the growth factors and/or cytokines can be accomplished by a variety of means such as spraying the graft or dipping the graft with a solution containing the growth factors and/or cytokines. Alternatively, the growth factors and/or cytokines can be incorporated into the graft matrix by mixing the growth factors and/or cytokines used in the preparation of the graft. In still other embodiments, semi-liquid matrices can be “staged” with growth factor or cytokines in an injectable form. For example, a first portion in a syringe or catheter may be a growth factor, while second portion (adjacent to the first in the syringe or catheter) that does not completely mix with the first portion may comprise a cell-biomaterial mixture. In similar fashion, different layers or stages may be sequentially administered to create gradients or preferential patterns of migration of the injected cells.

In other embodiments, different matrix compositions containing different combinations of growth factors and cytokines may be assembled together to form the desired graft. By selectively coating or providing a portion of the graft with growth factors and/or cytokines, specific cells can be preferentially recruited to different portions of the graft. Different portions of the graft can contain different combinations of growth factors and/or cytokines, resulting in the migration and preferential localization of different cell types on different portions of the graft, which translates to differential delivery to the target tissue.

In some embodiments, the number of cells incorporated is controlled to provide optimal cell survival within the biomaterial over time. For example, several embodiments using a hyaluronan biomaterial incorporate CDCs in a range from about 1000 CDCs/μl of hyaluronan to about 10,000 CDCs/μl of hyaluronan. In other embodiments, a CDC concentration of about 5000 CDCs/μl of hyaluronan is used. In some embodiments, a CDC concentration of about 1000 to about 3000 CDCs/μl, about 2000 to about 5000 CDCs/μl, about 4000 to about 7000 CDCs/μl, about 6000 to about 9000 CDCs/μl is used. In several embodiments, a CDC concentration of about 1000 to about 2000 CDCs/μl is used, including about 1000 to about 1500 CDCs/μl.

Several potential routes exist for delivery of the cell-biomaterial mixture to the heart, including, but not limited to: intracoronary infusion (antegrade via coronary arteries or retrograde via coronary veins), intramuscular injection (endocardially or epicardially), intravenous infusion, perfusion, and direct surface application. Choice of delivery route represents a balance between delivery efficiency, the invasiveness of the approach, off-target effects, and long-term benefits.

Catheter-based administration is used in some embodiments. Several catheters used to delivery the cell-biomaterial composition comprise specially designed needles to aid in delivery. For example, the Helix™ (BioCardia, Inc.) catheter comprises a distal needle with a corkscrew (helical) design capable of active fixation during injection which can help limit backflow from the needle track post-injection. Another example is the Myostar™ catheter (Cordis Corporation) which is used in conjunction with a NOGA® electromechanical mapping system which can identify regions of viable myocardium and enable targeted injections. In several embodiments, however, standard transendocardial or transepicardial catheters are used.

As described above, the viscosity of the final cell-biomaterial composition is controllable in various manners. Thus, in several, more viscous embodiments, the composition is able to be painted or placed directly onto the target cardiac tissue. In some embodiments, as described above, cells cultured on biomaterial sheets or within sponge or foam-like structures can be applied as a patch to the surface of the heart. In other, less viscous embodiments, cardiac perfusion may be used, delivering the cells to the heart by way of coronary vasculature. Intravenous infusion is used in still other embodiments. In several embodiments, direct intramuscular injection is used to deliver the composition directly to the heart tissue that needs to be repaired. In some embodiments, the cell-biomaterial composition is formulated to polymerize in situ after delivery.

In several embodiments, the biomaterial (or matrix) is administered (e.g., injected) to the crista terminalis, the right ventricular endocardium, the right ventricular septum, the septal or ventricle wall, atrium, the atrioventricular groove, or the right and left atrial appendages. In some embodiments, the matrix permits time released migration or diffusion of the cardiac stems cells (e.g., cardiospheres and/or CDCs) into the damaged heart.

### EXAMPLES

Examples provided below are intended to be non-limiting embodiments of the invention.

### Example 1

### Matrix Preparation & Cell Survival

Experiments to test the ability of cells to survive in biomaterials in culture were performed using hyaluronan-based hydrogel. Fibrin, alginate or other biomaterials may be used in addition to or instead of hyaluronan. Thiolated hyaluronan and polyethylene glycol diacrylate (PEGDA, a thiol-reactive crosslinker) were obtained as lyophilized solids. Warm (37° C.) degassed, deionized water was used to dissolve each component separately. Dissolution of the hyaluronan required about 30 minutes of rocking or shaking, while the PEDGA was readily solublized. Once reconstituted, the gel components were diluted in phosphate-buffered saline (pH=7.4). Cardiospheres, CDCs, cardiac stem cells, or mixtures thereof can be resuspended in any solution, and mixed with the hyaluronan solution. Hyaluronan (with or without cells) and PEGDA are then mixed in a 4:1 ratio to create a hydrogel. Gelation occurs within approximately 20 minutes. Diluting the hyaluronan or PEGDA components may be used to create a softer hydrogel. Diluting the PEGDA component by 50% approximately doubles the gelation time. Increasing the hyaluronan to PEGDA ratio (e.g., 8:1) also increases gelation time. Gelation also occurs more slowly as the pH is decreased, or as the mixture becomes more acidic.

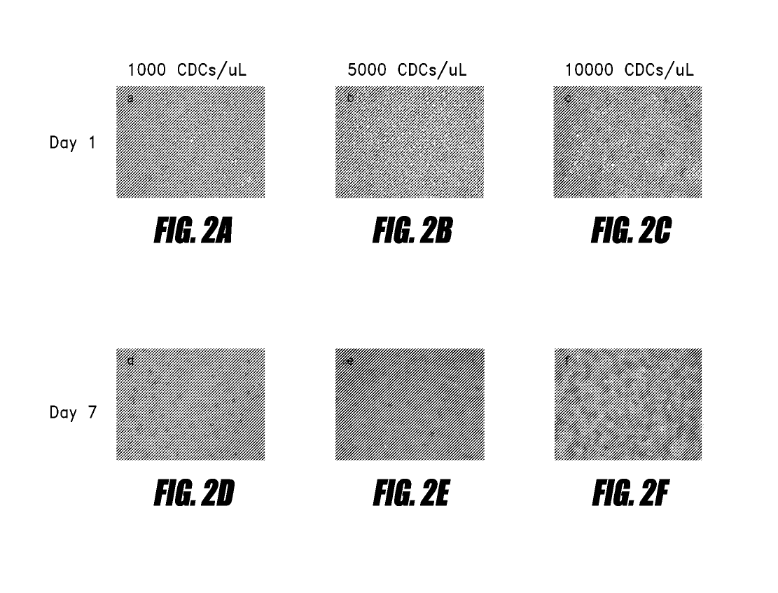

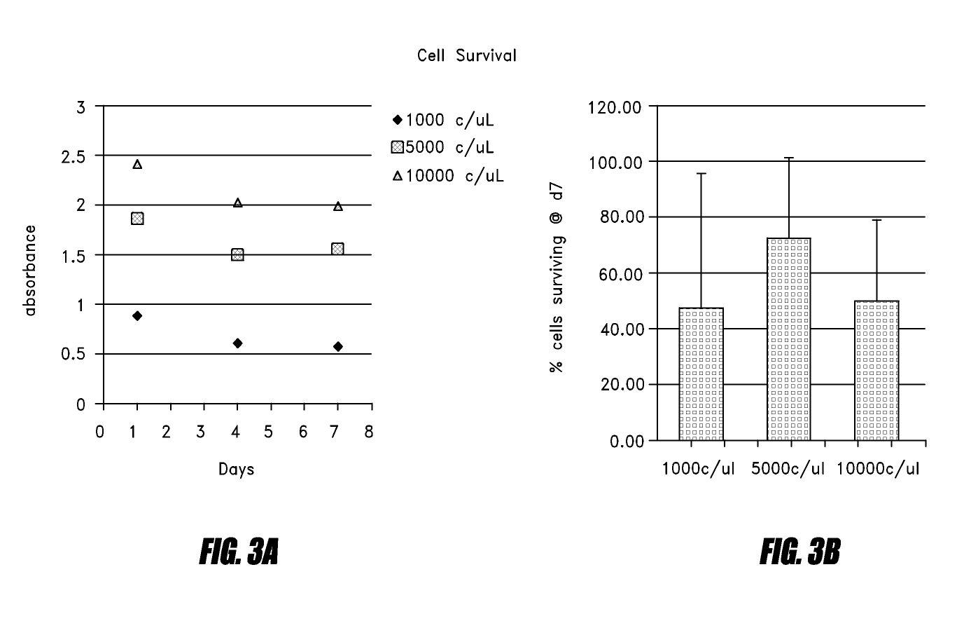

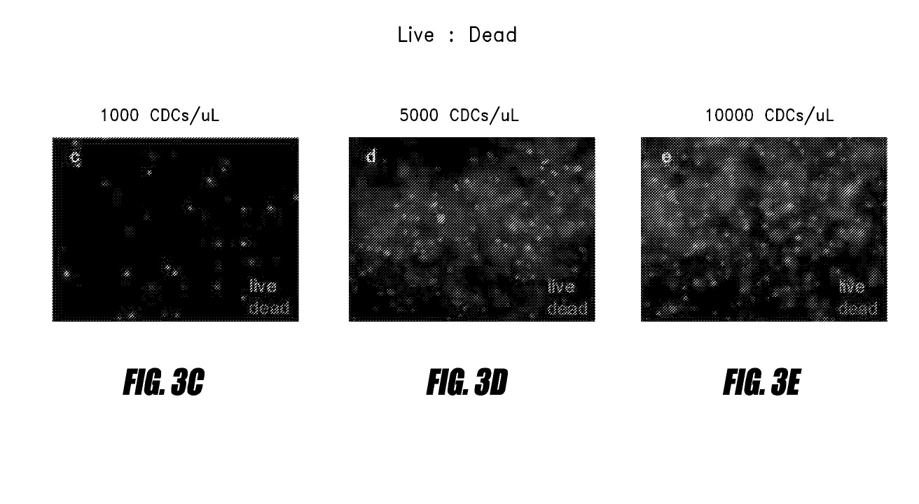

CDCs were incorporated into the aqueous hydrogel and allowed to set for about one hour to prevent CDCs from settling out of the gel. In other embodiments, cardiospheres will be incorporated into the aqueous hydrogel (or other biomaterial) in addition to, or instead of, the CDCs. Different concentrations of CDCs were incorporated into the hyaluronan. Survival was evaluated at 1 and 7 days post-incorporation. 45-70% of the cells incorporated into the hyaluronan survived, depending on the concentration. According to some other embodiments, cell survival rate may be increased. FIG. 2 shows bright field microscopic images of the various cell concentrations embedded in hyaluronan at Day 1 (a-c) or Day 7 (d-f) of culture. FIG. 3a depicts the absorbance signal read using the survival assay at 1 day, 4 days and 7 days of culture for each of the indicated cell concentrations. The percent of cells surviving at the end of the week relative to the beginning is shown in the graph FIG. 3b. The lowest cell concentration tested (1000 CDCs/μl), led to the lowest fraction of cells surviving, whereas the middle cell concentration (5000 CDCs/μl) led to the highest fraction of cells surviving (FIG. 3b). Cells remain round and dispersed at the end of one week at the lowest cell concentration (Compare FIGS. 2a to 2d). At the middle and high cell concentrations, networks and clusters of cells are seen at the end of one week (Compare FIGS. 2b to 2e and 2c to 2f, respectively). Cell-cell contact likely offers an important survival signal and enhances survivability in some embodiments. At the highest cell concentration, some slight overcrowding may have occurred.

The live to dead cell ratio was visualized using calcein and EthD-1 stain and correlated with the overall cell survival measured using the cell survival assay. An example preliminary analysis is depicted in FIG. 3c-e. A number of single dead cells can be seen at the lowest cell concentration, and some concentrated cell death can be seen in the cell clusters formed at the highest cell concentration.

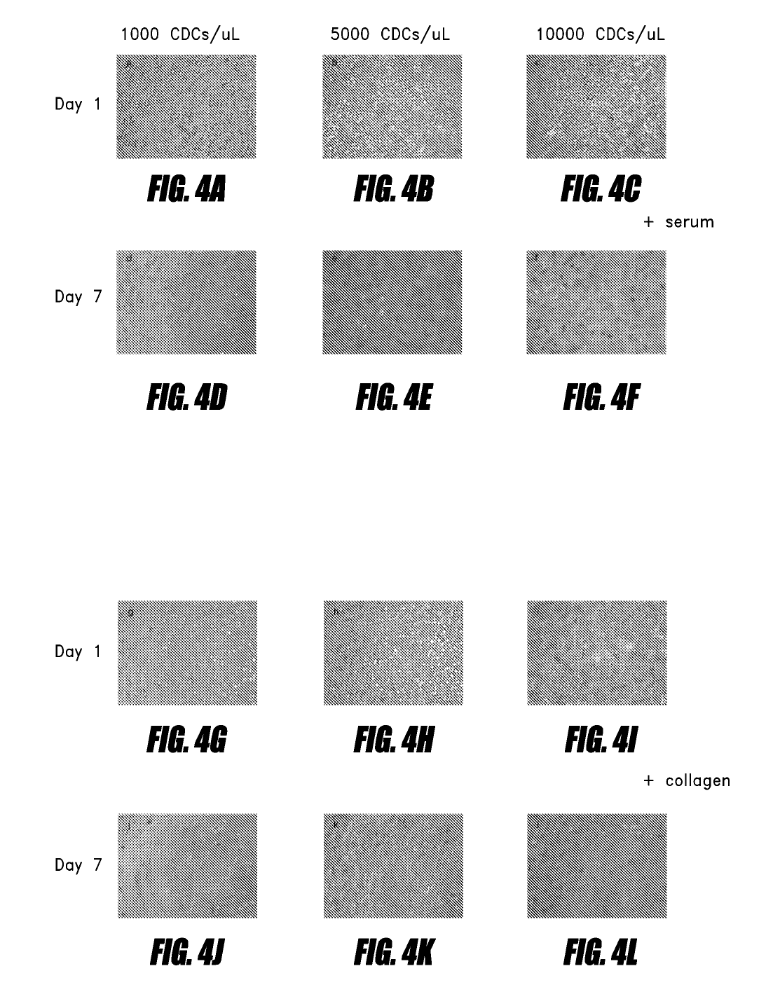

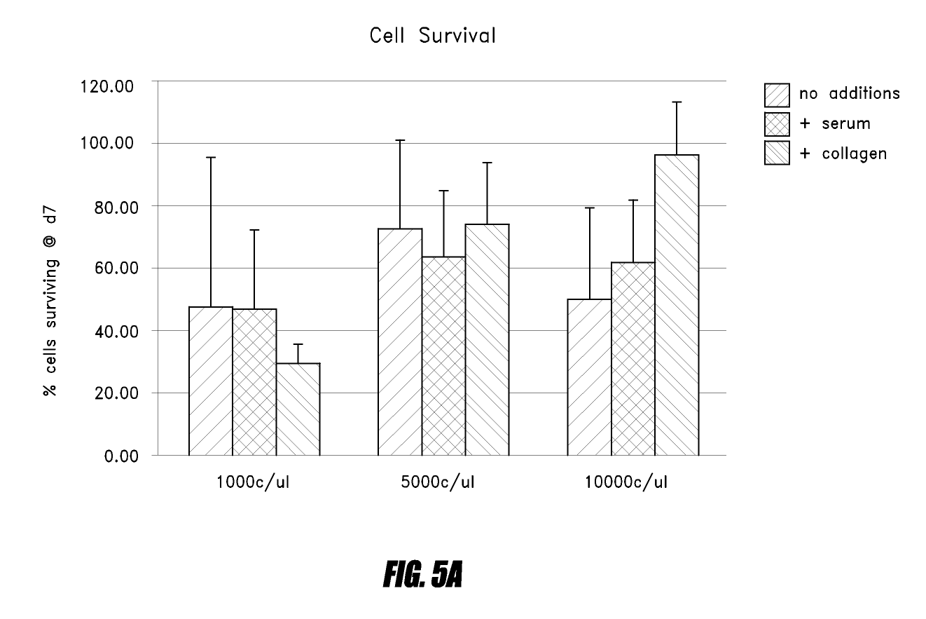

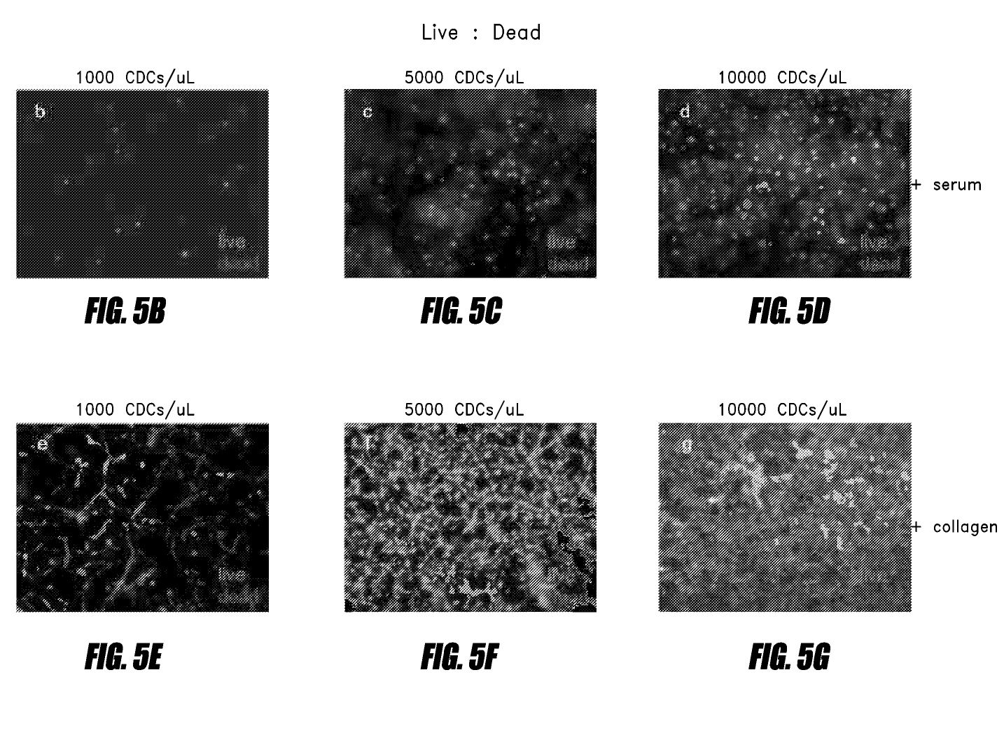

Supplementation of the hyaluronan biomaterial with either serum or collagen was also tested, as compared to hyaluronan alone. Different concentrations of CDCs were incorporated into hyaluronan supplemented with 7.4% serum or about 0.4% collagen. FIG. 4 depicts bright-field images of cells at Day 1 and Day 7 in culture. It was determined that 30-95% of cells survive over the course of 1 week in culture. (FIG. 5a) Cells remain round and dispersed at the end of one week at the lowest cell concentration in the serum condition. At the middle and high cell concentrations in the serum condition, networks and clusters of cells can be seen at the end of one week. In the collagen condition, a clear difference in cell morphology can be seen. Cells spread and network extensively within in the gel, particularly at the middle and high cell concentrations. The live to dead stain allows for even better visualization of cell morphology. (FIG. 5b-g). In general, the lowest and most variable levels of survival were seen at the lowest cell concentration. (FIG. 5a). At the middle cell concentration, CDCs cultured in either hyaluronan alone or hyaluronan supplemented with collagen showed an average cell survival rate of greater than 70%, although neither serum nor collagen as additives significantly improved the survival rate. At the highest cell concentration, the use of hyaluronan supplemented with collagen significantly improved cell survival when compared to hyaluronan alone, increasing from 72.5±28.8% to 96.3±17.1% (p<0.05), while the addition of serum to hyaluronan did not significantly affect survival. In some embodiments serum and/or collagen increases cell survival, while in other embodiments supplementation is not required. Optimal cell survival may not require supplementation of the hyaluronan in several embodiments, however, the difference in cell morphology seen with the addition of collagen may indicate a cell preference for the collagen-containing biomaterials, perhaps enhancing cell survival in hypoxic conditions or enhancing cell migration capacity.

The following assays may be used in the Examples disclosed herein.

Cell Survival Assay: Cell survival over the course of 1 week was assessed for cells embedded in the various gel formulations. Cells were incorporated into the biomaterial as described and the gel was cast in a 96-well plate. After gelation, normal cell culture media was added to each well. A cell counting kit (Cell Counting Kit-8, Dojindo), which utilizes a water-soluble tetrazolium salt, WST-8 [2-(2-methoxy-4-nitrophenyl)-3-(4-nitrophenyl)-5-(2,4-disulfophenyl)-2H-tetrazolium, monosodium salt], was utilized. WST-8 produces a media-soluble formazan dye upon reduction in the presence of an electron carrier (e.g., by dehydrogenases in cells). The amount of the formazan dye generated by the activity of dehydrogenases in cells is directly proportional to the number of living cells. At 1-3 day intervals over the course of 1 week, 20 μL of premixed WST-8 was added to each test well and incubated for 30 minutes. The absorbance was read on a SpectraMax M5 Microplate Reader (Molecular Devices), one hour after the start of incubation. Each reading was normalized to a day 1 reading in order to express the percent survived at each time point. The percent survived represents cumulative cell proliferation and cell death.

Live Dead Cell Assay: The ratio of live to dead cells was assessed using a viability and cytotoxicity kit (LIVE/DEAD® Viability/Cytotoxicity Kit, Invitrogen). This kit provides a two-color fluorescence cell viability assay that is based on the simultaneous determination of live and dead cells with two probes that measure recognized parameters of cell viability. Live cells were distinguished by the presence of ubiquitous intracellular esterase activity, determined by the enzymatic conversion of the virtually nonfluorescent cell-permeant calcein AM to the intensely fluorescent calcein. Calcein is known to be well retained within live cells, producing an intense uniform green fluorescence in live cells (ex/em ˜495 nm/˜515 nm). EthD-1 enters cells with damaged membranes and undergoes a 40-fold enhancement of fluorescence upon binding to nucleic acids, thereby producing a bright red fluorescence in dead cells (ex/em ˜495 nm/˜635 nm). EthD-1 is excluded by the intact plasma membrane of live cells. Cells embedded in the various gel formulations were labeled at the end of the 1 week culture period. Each cell-biomaterial formulation was washed with PBS to remove excess serum. Calcein AM (2 μM diluted in PBS) and EthD-1 (4 μM diluted in PBS) was added to the gels which were incubated for 30 minutes. At the end of the incubation period, the cells embedded within the gels were examined using an Eclipse TE2000-U (Nikon) fluorescence microscope with Image-Pro Plus software (Media Cybernetics). Images were captured of each gel condition and the number of live and dead cells counted (with at least 200 total cells counted per condition). The live to dead cell ratio was then calculated.

### Example 2

### CDC Survival at 72 Hours in Hyaluronan Hydrogels With or Without Collagen

In order to further characterize the survival of cells in various matrices, hyaluronan-based hydrogels were reconstituted as described above. CDCs were incorporated into the hydrogels during the crosslinking process (prior to gelation) and seeded on a 96 well plate. The final aqueous cell solution passed readily through a 30-gauge needle.

Plates were pre-coated with a thin layer of gel prior to seeding the CDC-hydrogels. This was done to prevent cells from settling out of the gel during the time needed for gelation and coming into contact with the polystyrene tissue culture plates. Gelation occurred within approximately 20 minutes. In several embodiments, a 20 minute gelation time is rapid enough for the hydrogel to improve cell retention within the myocardium yet also slow enough to allow for complete passage of the cell-biomaterial solution to pass fully through the delivery mechanisms (e.g., catheters, needles, etc.). Longer or shorter gelation times are used in some embodiments. As discussed above, variations in the concentration of the crosslinker, thiol-modified PEGDA, and the amount of PBS can be readily made to develop a faster or slower gelling formulation.

After the hyaluronan-cell compositions set, media was added on top of the gels. CDCs were first tested in hyaluronan alone and hyaluronan supplemented with 0.4% collagen at different cell concentrations (100, 1000, 5000 and 10,000 cells/μL). In some embodiments, cell concentrations are between about 500-2000 cells/μL), between about 750-1500 cells/μL, or between about 900-1250 cells/μL. Depending on the potency and viability of a given population of isolated CDCs, greater or lesser concentrations of cells may be used in several embodiments. For example, in some embodiments, between about 5000-10000 cells/μL are used. In some embodiments, between about 3000-7000 cells/μL are used.

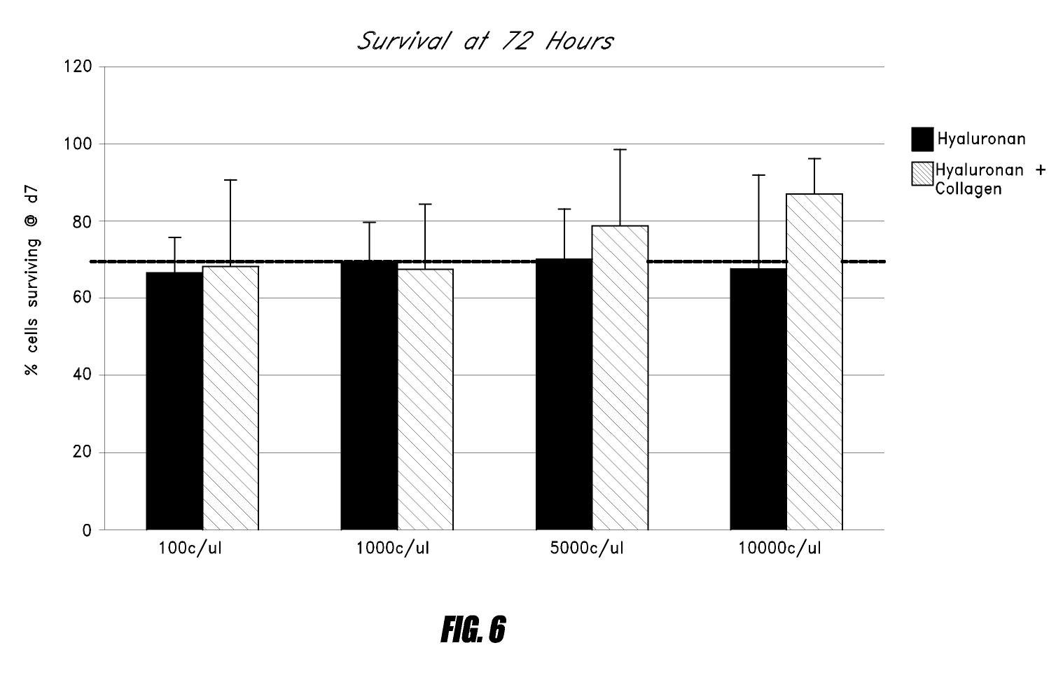

FIG. 6 depicts CDC viability relative to the baseline viability level, as measured by the in vitro cell viability assay 72 hours post-seeding at each of the cell concentrations. For all cell concentrations, the average viability at the end of 72 hours was ˜70% or greater. These results suggest that the local environment plays an important role in the survival of the cells, as culturing CDCs outside the normal serum-rich in vitro culture environment led to a limited amount of apoptosis (e.g., survival <100%), rather than proliferation. These results also indicate that CDCs are equally as compatible with hyaluronan and collagen supplemented hyaluronan. The 72 hour time point was chosen as an intermediate time-point (as compared to Example 1) and is used in the art as a time point approximating the amount of time required for the majority of embedded cells to migrate from a matrix and engraft under in vivo conditions.

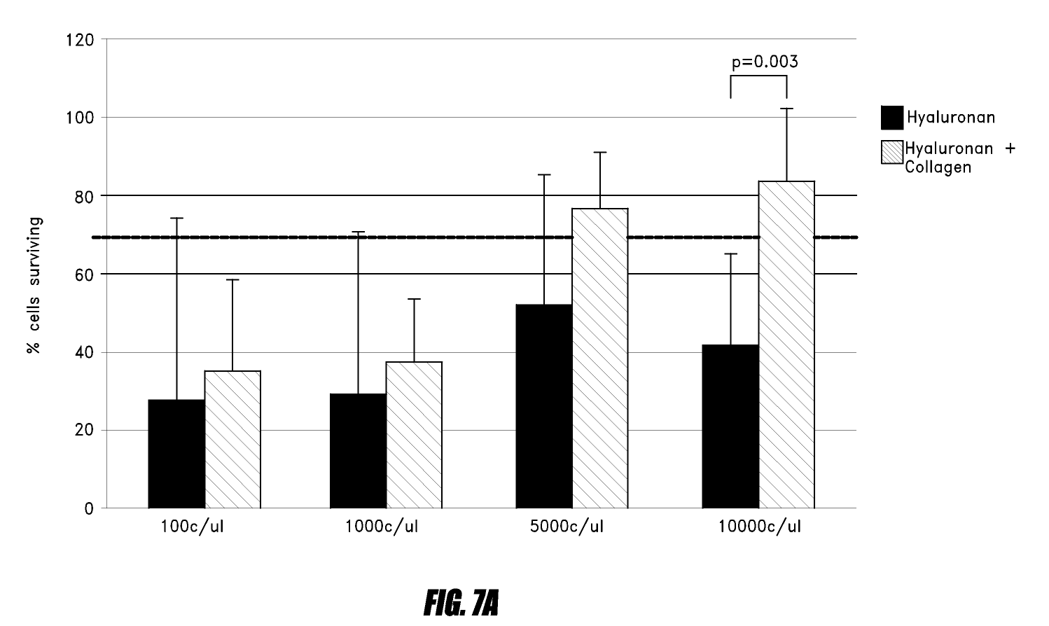

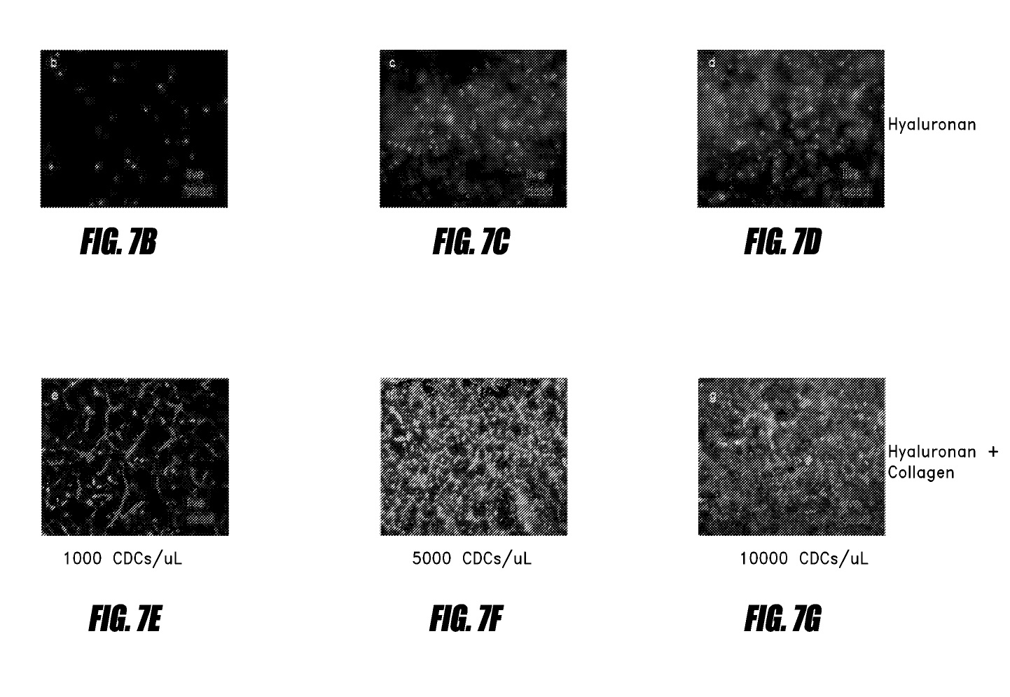

Building on the data described in Example 1, FIG. 7 illustrates the level of CDC survival detected one week post-seeding in hyaluronan or collagen-supplements hyaluronan. One week is an approximation of the time at which hydrogels may begin to be biodegraded in vivo. Notably though, 1 week is a long-term in vitro readout for this type of survival assay. A significant difference between hyaluronan alone versus collagen supplemented hyaluronan was detected at the highest cell concentration, with collagen supplemented hyaluronan maintaining ˜80% CDCs as viable while cells embedded in hyaluronan alone survive at about a 40% level. (FIG. 7a) Live-dead staining of CDCs within the hydrogels was performed to confirm the quantitative viability findings and assess CDC morphology (FIG. 7b-g). As discussed above, viable cells fluoresce green due to cleavage of acetomethoxy-calcein (calcein AM) by intracellular esterases while non-viable cells fluoresce red due to binding of ethidium homodimer to nucleic acids which occurs due to loss of cell membrane integrity. At the lowest cell concentration, the cells in hyaluronan alone remain round and dispersed. At the middle and high cell concentrations, clusters of round live cells are seen. The rounded morphology, may be due to the relative lack of any cell adhesion sites presented by the hydrogel. (FIG. 7b-d).

In contrast, cells within the collagen supplemented hyaluronan hydrogels are elongated, which represents a more native morphology. Thus the addition of collagen appears to affect both cell morphology and cell survival over the course of 1 week. The difference in cell morphology seen with the addition of collagen may indicate a cell preference for the collagen-containing hyaluronan. (FIG. 7e-g). This may, in some embodiments, allow for enhanced cell survival during in vitro hypoxia studies and/or within the ischemic myocardium. The morphological differences may also correspond to differences in cell phenotype, especially in expression of cell adhesion molecules

Interestingly, a significant difference in cell survival between the biomaterials was detected only at the 10000 CDC/uL cell concentration. (FIG. 7a). This suggests that at higher cell population densities, a unique, pro-survival, local environment is created within the biomaterial supplemented with collagen. As discussed above, this may be due to the ability of the collagen to bind and slowly re-release over time growth factors or other molecules released from the cells incorporated into the biomaterial. However, as shown in FIGS. 5a and 7a, hyaluronan alone (5a and 7a) and hyaluronan supplemented with serum (5a) show no significant difference in cell survival as compared to hyaluronan supplemented with collagen at lower cell concentrations.

Moreover, the survival of cells at varying concentrations at 72 hours did not differ based on the type of hydrogel (FIG. 6) and survival was ˜70% for all groups tested. These results suggest that the un-supplemented biomaterial alone has the capacity to support cell survival (including possible binding and re-release of growth factors or other molecules released from the cells incorporated into the biomaterial) at all concentrations for at least 72 hours and at several cell concentrations for up to 1 week. Thus, depending on the concentration of cells needed for a particular application, either a hyaluronan alone, or a supplemented hyaluronan biomaterial may be used. Hyaluronan alone presents several advantages for use in humans (particularly in a 72-hour time window), as the biomaterial is xeno-free. However, the collagen data do not preclude the use of higher (e.g., 10000 cells/uL) cell concentrations with a supplemented hyaluronan in humans, though they do indicate that survival at 1 week deserves consideration when designing the therapeutic regimen, as it may impact the overall efficiency of cell delivery to a target tissue of a patient.

### Example 3

### Migration Assay

As cells incorporated into a biomaterial likely have to migrate from the biomaterial in order to provide the most efficacious repair or regeneration of damaged tissue, the present experiment was designed to evaluate the in vitro migratory potential of cells incorporated within the hyaluronan and collagen-supplemented hyaluronan hydrogels. While either cardiospheres, CDCs or other cells types may be delivered via a biomaterial, in this experiment, CDCs were labeled with calcein to enable the tracking of migrating cells. Cell labeling was performed by established techniques. Calcein-labeled CDCs were incorporated into various hydrogels at a cell concentration of 10,000 cells/μL. CDCs within the hydrogels were cultured in a transwell plate setup that allowed for cell migration from the upper chamber, through pores in the bottom of the chamber insert, and into the lower chamber where they could be detected. Fetal bovine serum was used as a chemoattractant in the lower chamber. CDCs plated directly in the transwell without hydrogel were used as a control.

Labeled cells emit a fluorescent signal that was detected using a plate reader which reads fluorescence from the bottom of the plate, thereby quantifying the number of cells that migrate to the lower chamber. Each experiment was performed in triplicate to allow statistical comparison of the results Cell migration out of the gel and toward a chemoattractant in the lower chamber was monitored over the course of 72 hours.

It was hypothesized that a biomaterial matrix that is less viscous, and thus more porous, would allow for higher migration of the cells out of the material. Likewise, it was hypothesized that those biomaterials that support the higher cell survival percentages would have higher migration rates, as a higher percentage of the cells contained therein may be viable and thus capable of responding to a chemoattractant signal.

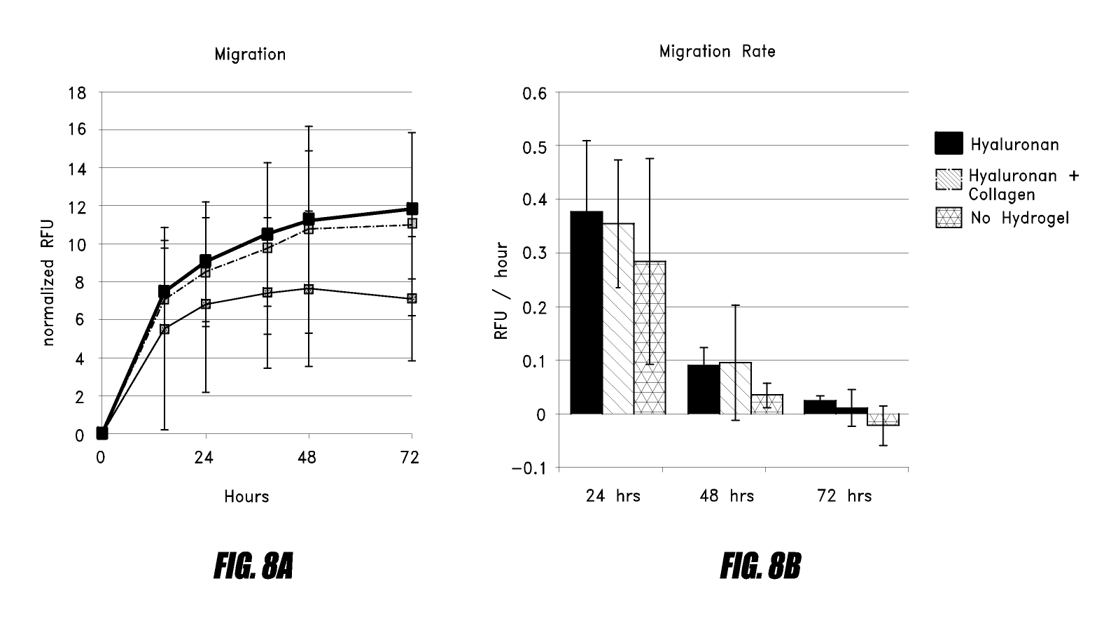

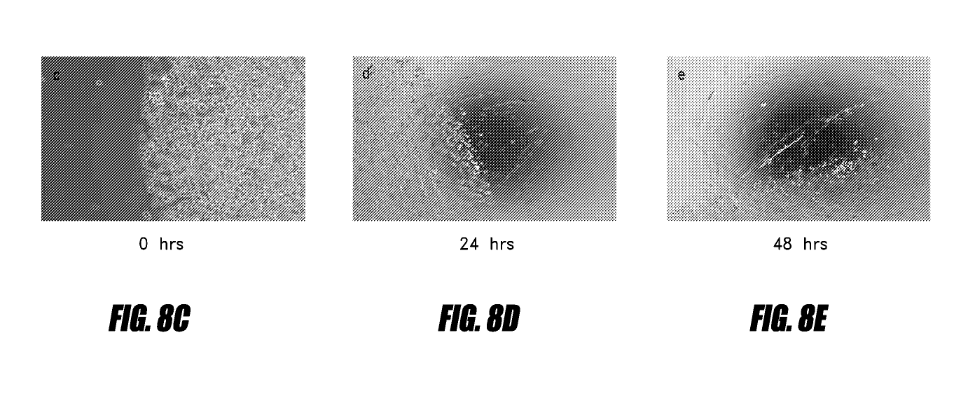

As shown in FIG. 8a, CDCs migrated out of the various hydrogels and into the lower chamber as readily as control cells. Maximal migration rate for all conditions was observed within the first 24 hours of the assay (see FIG. 8b). After 24 hours, the rate of migration decreased in both varieties of hyaluronan and control experiments. This may be due to time-dependent loss of the in vitro serum gradient or may be due to overcrowding of migrated cells within the lower chamber. In contrast to the hypothesis, the data collected to date unexpectedly indicated that the rate of migration out of the various hydrogels is not significantly different as compared to control migration. Further, the rate of migration of CDCs out of the hyaluronan alone as compared to the collagen supplemented hyaluronan was unexpectedly similar with no significant difference observed at any of the time points analyzed to date. While these results are contrary to the initial hypotheses, they reinforce the concept that hyaluronan alone and collagen supplemented hyaluronan are equally as effective with respect to the release of incorporated cells into a target tissue. Thus, in some embodiments, the “grasp” of the matrix is one-way or unidirectional, in that the cells are initially retained within the matrix, but are not later inhibited from migrating out of the matrix. Such a unidirectional retention is particularly beneficial in several embodiments, in that the cells are well retained in the matrix during and immediately post-delivery (discussed below), but are not inhibited from migrating out of the matrix and/or engrafting into the target tissue.

Further to the transwell migration assay, additional experiments were performed that allowed for the direct observation of CDC migration. Cells were incorporated into the hyaluronan hydrogel as described above and then spotted onto a fibronectin-coated plate. The cell-hydrogel spot could be observed with a well-defined edge at the beginning of the culture period (see FIG. 8c). Within 24 hours of culture, cells could be seen migrating onto the fibronectin-coated plate (FIG. 8d). After 48 hours, the plate was filled with CDCs that had migrated out of the hydrogel (FIG. 8d).

### Example 4

### In Vivo Engraftment of CDCs

In order to demonstrate that a cell-hydrogel formulation can be delivered in a targeted fashion via direct injection, myocardial infarction was created in several mice and cells in PBS, hyaluronan alone, or collagen-supplemented hyaluronan were delivered by injection through a 30-gauge needle. Severe Combined Immune Deficiency (SCID) mice were used in order to eliminate any impact of host rejection of the transplanted human CDCs on the results. CDCs were labeled with CellTracker™ CM-DiI (as described above) to enable visualization of transplanted cells. Immediately prior to injection, CDCs were suspended at 10000 cells/μL in either PBS, hyaluronan, or collagen-supplemented hyaluronan, and 1.5×10⁵ cells were delivered at two sites within the border zone of the infarct. Because CDCs were added to the hydrogels immediately prior to injection gelation was primarily in situ.

Euthanasia was performed 24 hours post-injection and the hearts collected for analysis by polymerase chain reaction (PCR) and/or histological examination. Quantitative PCR for human Alu sequences (a repetitive element in the human genome) was used to calculate the percentage of cells engrafted in each animal. Well-known nucleic acid isolation and PCR techniques were used. CDCs from a single cell line (e.g., isolated and expanded together from a single donor) were used, thereby enabling the creation of a single standard curve and reducing the potential for variation due to potency of a given line.

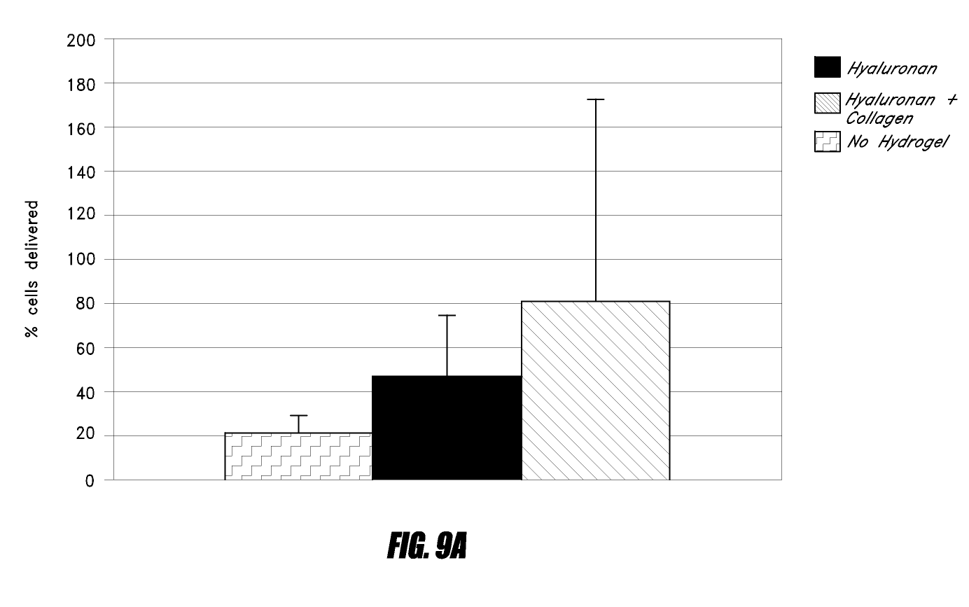

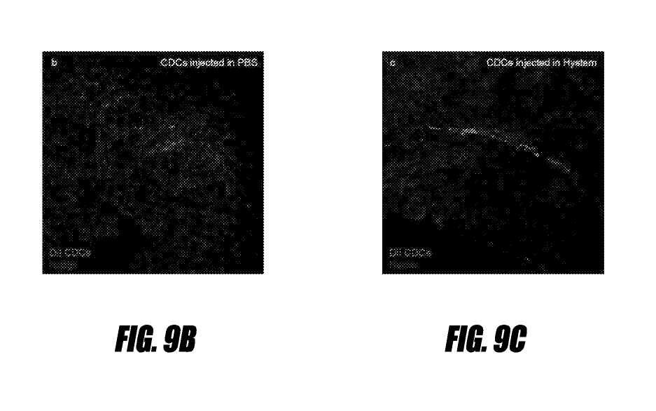

As shown in FIG. 9a, CDCs delivered in PBS showed ˜20% engraftment 24 hours after delivery. In contrast, CDCs delivered in hyaluronan averaged ˜50% engraftment, thus more than doubling the engraftment of cells delivered in a simple liquid vehicle. CDCs delivered in collagen-supplemented hyaluronan demonstrated ˜80% engraftment after 24 hrs. Thus, in several embodiments, hydrogels of either variety are provided to improve engraftment as compared to a PBS-alone delivery vehicle.

Fluorescent histological analysis revealed that that CDCs delivered in PBS (FIG. 9b) were dispersed throughout the myocardium. The cell nuclei are Hoeschst-labeled (blue) and the DiI-labeled CDCs appear red. Cardiac tissue lacks significant interstitial space to house injected cells. Thus, when targeting cardiac tissue with cells in a liquid vehicle, a significant portion of cells administered are likely to be washed away via the turbulence of blood flow. Additionally, the repetitive compressive effect of the contracting heart muscle tends to physically force cells injected in a liquid media out of the limited space between myocytes.

In contrast to PBS-based delivery, CDCs delivered in hyaluronan remained within a defined area resembling the needle track (see FIG. 9c). Thus, in several embodiments, direct intramyocardial injection of CDCs in the hyaluronan hydrogel helps localize cells at the injection site, despite the fluid and compressive forces described above. In one embodiment, the viscosity of the hyaluronan biomaterial provides the short-term benefit of retaining the cells at or substantially near the site of injection in the target tissue. In other words, in some embodiments, the higher viscosity of the matrix increases the retention of cells at the injection site. In one embodiment, the matrix also provides an additional benefit in enhancing the engraftment of the cells in the target tissue. This may, in some embodiments, be due to the matrix providing an environment in which the cells can survive for extended periods of time in vivo. Thus, in contrast to a PBS (or other liquid media) a higher degree of viable cells are present post-injection, thus enhancing the engraftment of the cells into the heart tissue by holding the cells in juxtaposition to the target tissue for a sufficient time for a therapeutically effective amount of cells to engraft.

In accordance with the examples, in several embodiments, CDCs are highly compatible with hydrogels alone as well as with supplemented hydrogels. CDCs were shown to have survive within the hydrogel formulations but also demonstrate a capacity to migrate out of the hydrogels. The cell concentrations tested may also allow for delivery of high doses of CDCs within relatively small volumes of a hydrogel. Further, pursuant to the data, in several embodiments, the CDC-hydrogel formulation, when delivered via direct intramyocardial injection, enables cell retention within the desired region and/or increases cell engraftment following delivery. In some embodiments, hydrogel-cell formulations are deliverable through catheter delivery systems and results in improved targeted delivery, engraftment, and repair of cardiac tissue.

### Example 5

### Hypoxia

To expand on the discoveries made in the above experiments, human cardiospheres or CDCs will be subjected to a hypoxic environment in order to examine cell survival in the face of hypoxia. Cell survival will be examined over the course of 1 week for cells embedded in the various biomaterials. Cells will be incorporated in the biomaterial and the biomaterial will be cast in a 96-well plate. After gelation has occurred, normal cardiosphere or CDC media will be added to each well. A day 1 reading using the cell survival assay will be taken while cells remain in a normoxic environment. Cells will then be placed in a hypoxia incubator with 1% oxygen, 5% carbon dioxide, and balance nitrogen gas environment. This will simulate the potentially hypoxic environment that the administered cells would be placed into in vivo when administered to a patient suffering from an adverse cardiac event. Cells will be subjected to 7 days of hypoxia and will then be assessed. Each reading will be normalized to the day 1 reading in order to express the percent survived at the end of one week. Each biomaterial will then be examined using the live dead cell assay. Each experiment will be performed in triplicate. An average and standard deviation will then be calculated so that the different experimental conditions can be statistically compared.

Various modifications and applications of embodiments of the invention may be performed, without departing from the true spirit or scope of the invention. Method steps disclosed herein need not be performed in the order set forth. It should be understood that the invention is not limited to the embodiments set forth herein for purposes of exemplification, but is to be defined only by a reading of the appended claims, including the full range of equivalency to which each element thereof is entitled.

1. A method of facilitating targeted delivery of cardiac stem cells and enhancing engraftment of said cells to repair or regenerate cardiac tissue, the method comprising:
obtaining cardiosphere-derived cells,
wherein said cells are obtained from healthy mammalian non-embryonic cardiac tissue;
combining said cells with a biocompatible hydrogel to generate a matrix,
wherein said hydrogel comprises cross-linked hyaluronan;
injecting said matrix into a subject to repair or regenerate cardiac tissue,
wherein said matrix is adapted to initially retain said cells upon injection, to promote the survival of said cells, and to subsequently allow release and migration of said cells from said matrix to a targeted cardiac tissue,
wherein said matrix facilitates the targeted delivery of the cells to said targeted cardiac tissue over time in vivo by reducing extraneous migration of said cells to non-targeted locations, and
wherein said matrix enhances engraftment of said cells into the heart, thereby repairing or regenerating said cardiac tissue.

2. The method according to claim 1, wherein the hydrogel further comprises collagen, wherein said collagen results in preferential attachment of said cardiosphere-derived cells to said matrix.

3. The method according to claim 2, wherein said collagen is thiolated collagen.

4. (canceled)

5. The method according to claim 1, wherein the matrix facilitates the retention of injected cells at the site of injection in the targeted cardiac tissue for at least twenty-four hours post-injection.

6. The method according to claim 1, wherein said matrix is adapted to promote the survival of about 60% of said cells within said matrix for a period of at least 72 hours.

7. The method according to claim 1, wherein the matrix is adapted to allow maximal migration rates of said cells out of the matrix to the targeted cardiac tissue for a period of at least about twenty-four hours post-injection.

8. The method according to claim 1, wherein said sample of healthy mammalian cardiac tissue is obtained from said subject.

9. The method according to claim 1, wherein said sample of healthy mammalian cardiac tissue is obtained from a mammal other than said subject.

10.-11. (canceled)

12. The method according to claim 1, wherein the number of cells combined with said matrix is from about 1000 to about 10000 cells per microliter of matrix.

13.-14. (canceled)

15. The method according to claim 1, wherein said injection comprises injection of the matrix to the heart of said subject.

16.-18. (canceled)

19. The method according to claim 1, wherein said hyaluronan is crosslinked with polyethylene glycol diacrylate.

20. The method according to claim 19, wherein the polyethylene glycol diacrylate is combined with said hyaluronan in a ratio of between about 16 parts hyaluronan to 1 part polyethylene glycol diacrylate to about 1 part hyaluronan to 1 part polyethylene glycol diacrylate.

21. (canceled)

22. The method according to claim 19, wherein the ratio of hyaluronan to polyethylene glycol diacrylate results in a gelation time of between approximately 1 minute to approximately 60 minutes.

23.-26. (canceled)

27. A system for repair or regeneration of cardiac tissue comprising:
isolated cardiosphere-derived cells;
a cell-containing matrix; and
a delivery device,
wherein cardiosphere-derived cells are isolated from healthy mammalian non-embryonic cardiac tissue,
wherein said matrix comprises a biocompatible hydrogel comprising cross-linked hyaluronan,
wherein said isolated cardiosphere-derived cells are incorporated into said cross-linked hyaluronan to generate said matrix,
wherein said matrix is biocompatible and suitable for in vivo administration to the cardiac tissue of a subject;
wherein said matrix is adapted to initially retain said cells upon administration, to promote survival of said cells, and to subsequently allow release and migration of said cells from said matrix to a targeted cardiac tissue,
wherein said matrix facilitates the targeted delivery of the cells to the cardiac tissue over time in vivo by reducing extraneous migration of said cells to non-targeted locations; and
wherein said matrix enhances engraftment of said cells into the cardiac tissue.

28. The system of claim 27 wherein said cardiosphere-derived cells are incorporated into said biocompatible biomaterial in a concentration of about 1000 to 10000 cells per microliter of biomaterial.

29. The system of claim 27, wherein said cardiosphere-derived cells are isolated from cardiac tissue obtained from said subject.

30. The system of claim 27, wherein said cardiosphere-derived cells are isolated from cardiac tissue obtained from a mammal other than said subject.

31. The system of claim 27, wherein said hydrogel further comprises collagen.

32. The system of claim 31, wherein said wherein said collagen is thiolated collagen.

33.-44. (canceled)

45. A method of facilitating targeted delivery of cardiac stem cells to repair or regenerate cardiac tissue, the method comprising:
obtaining a plurality of cardiac stem cells,
wherein said cells are obtained from healthy mammalian non-embryonic cardiac tissue obtained from a first subject;
combining said cells with a biocompatible hydrogel comprising cross-linked hyaluronan to generate a cell-seeded matrix;
administering said cell-seeded matrix to a second subject to repair or regenerate cardiac tissue,
wherein said cell-seeded matrix initially retains said cardiac stem cells upon administration to said second subject and subsequently allows release of at least a portion of said cardiac stem cells from said cell-seeded matrix and migration to a targeted cardiac tissue;
wherein said initial retention facilitates the targeted delivery of said cardiac stem cells to said targeted cardiac tissue by reducing extraneous migration of said cardiac stem cells cells to non-targeted locations, thereby repairing or regenerating said cardiac tissue.
